(19) United States
(12) Patent Application Publication
(10) Pub. No.: US 2017/0215941 A1
(43) Pub. Date: Aug. 3, 2017

(54) MEDICAL TREATMENT DEVICE

(71) Applicant: OLYMPUS CORPORATION, Tokyo (JP)

(72) Inventor: Hiroki KAZUNO, Tokyo (JP)

(73) Assignee: OLYMPUS CORPORATION, Tokyo (JP)

(21) Appl. No.: 15/492,668

(22) Filed: Apr. 20, 2017

Related U.S. Application Data
(63) Continuation of application No. PCT/JP2014/078077, filed on Oct. 22, 2014.

(51) Int. Cl. A61B 18/14; A61B 18/12

(52) U.S. Cl. CPC A61B 18/14; A61B 18/1447; A61B 18/1206; A61B 2018/145; A61B 2018/0063

(57) ABSTRACT

A medical treatment device includes a pair of holding members for opening and closing about an axis in accordance with an operation of an operating unit, and a shaft having one end connected to the operating unit and having the other end to axially support the holding members through the axis so as to allow the holding members to open and close. One of the holding members includes an energy application portion for contacting a body tissue to apply energy to the body tissue with the body tissue grasped by the holding members. The energy application portion is connected to a flexible substrate to be electrically connected to a lead wire provided inside the shaft extending from the other end of the shaft toward the one end of the shaft. The flexible substrate is connected to the lead wire closer to the one end of the shaft than the axis.

### CROSS REFERENCES TO RELATED APPLICATIONS

This application is a continuation of PCT international application Ser. No. PCT/JP2014/078077, filed on Oct. 22, 2014 which designates the United States, incorporated herein by reference.

### BACKGROUND

1. Technical Field

The disclosure relates to a medical treatment device.

2. Related Art

In recent years, medical treatment devices have been known that apply energy to body tissue to perform treatment on the body tissue (for example, see JP 2003-325537 A).

A medical treatment device disclosed in JP 2003-325537 A includes a pair of treatment tool pieces which is rotatably connected to each other by a pivot shaft. Here, distal ends of the pair of treatment tool pieces are opened and closed when the pair of treatment tool pieces is rotated about the pivot shaft and a body tissue can be grasped by the distal ends. Further, a plurality of heating elements is embedded in the distal end of one treatment tool piece of the pair of treatment tool pieces. Then, in the medical treatment device, the body tissue is grasped by the distal ends of the pair of treatment tool pieces and power is supplied to the plurality of heating elements to apply heat energy to the body tissue in order to treat the body tissue.

Here, in the medical treatment device disclosed in JP 2003-325537 A, a flexible substrate is disposed inside one treatment tool piece and power supplied from the outside is transmitted to the plurality of heating elements by the flexible substrate. That is, in the medical treatment device disclosed in JP 2003-325537 A, since the flexible substrate is used as a wiring that transmits power from the outside to the plurality of heating elements instead of a lead wire, a small-sized medical treatment device can be achieved.

### SUMMARY

In some embodiments, a medical treatment device includes a pair of holding members configured to open and close about a first rotation axis in accordance with an operation of an operating unit, and a shaft having one end connected to the operating unit and having the other end to axially support the pair of holding members through the first rotation axis so as to allow the pair of holding members to open and close. At least one of the pair of holding members includes an energy application portion configured to contact a body tissue to apply energy to the body tissue with the body tissue grasped by the pair of holding members. The energy application portion is connected to a flexible substrate configured to be electrically connected to a lead wire provided inside the shaft extending from the other end of the shaft toward the one end of the shaft to supply energy to the energy application portion. The flexible substrate is connected to the lead wire at a location closer to the one end of the shaft than the first rotation axis.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram schematically illustrating a medical treatment system according to a first embodiment of the invention;

FIG. 2 is a diagram illustrating a distal end part of the medical treatment device illustrated in FIG. 1;

FIG. 3 is a diagram illustrating the distal end part of the medical treatment device illustrated in FIG. 1;

FIG. 4 is a diagram illustrating the distal end part of the medical treatment device illustrated in FIG. 1;

FIG. 5A is a diagram illustrating a flexible substrate;

FIG. 5B is a diagram illustrating the flexible substrate;

FIG. 6A is a diagram illustrating an operation of opening and closing first and second holding members;

FIG. 6B is a diagram illustrating an operation of opening and closing the first and second holding members;

FIG. 7 is a diagram illustrating Modified Example 1-1 of the first embodiment of the invention;

FIG. 8 is a diagram illustrating Modified Example 1-2 of the first embodiment of the invention; and

FIG. 9 is a cross-sectional view illustrating a configuration of a shaft body according to a second embodiment of the invention.

### DETAILED DESCRIPTION

Exemplary embodiments of the invention will be described below with reference to the drawings. The embodiments to be described below do not limit the invention. The same reference signs are used to designate the same elements throughout the drawings.

### First Embodiment

Schematic Configuration of Medical Treatment System

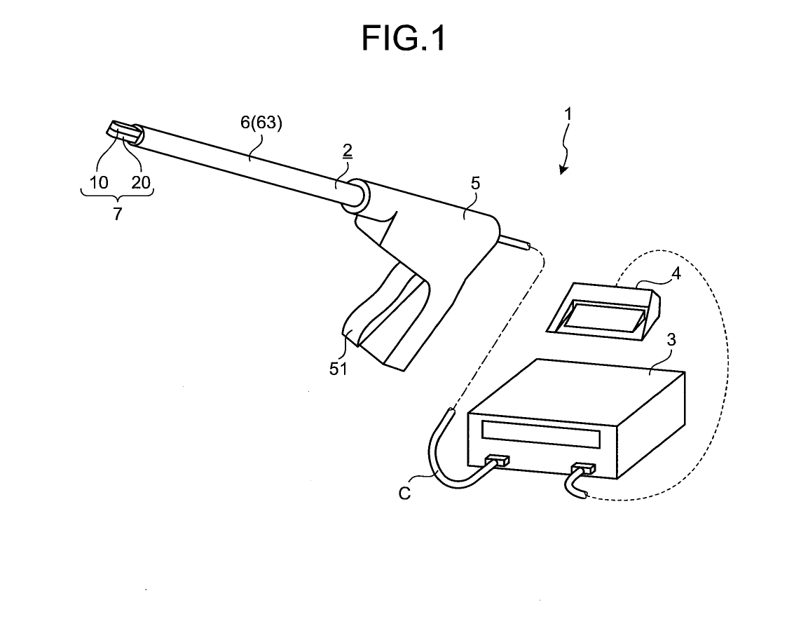

FIG. 1 is a diagram schematically illustrating a medical treatment system 1 according to a first embodiment of the invention.

The medical treatment system 1 is used to perform a treatment (for example, a blood vessel sealing or dissecting) on a body tissue by applying energy to a body tissue which is a treatment object. As illustrated in FIG. 1, the medical treatment system 1 includes a medical treatment device 2, a control device 3, and a foot switch 4.

Configuration of Medical Treatment Device

The medical treatment device 2 is, for example, a linear type surgical treatment tool which is used to perform a treatment for a body tissue through an abdominal wall. As illustrated in FIG. 1, the medical treatment device 2 includes an operation handle (an operating portion) 5, a shaft 6, and a grasping portion 7.

The operation handle 5 is a portion which is gripped by an operator. Then, as illustrated in FIG. 1, the operation handle 5 is provided with an operation knob 51.

Configuration of Shaft

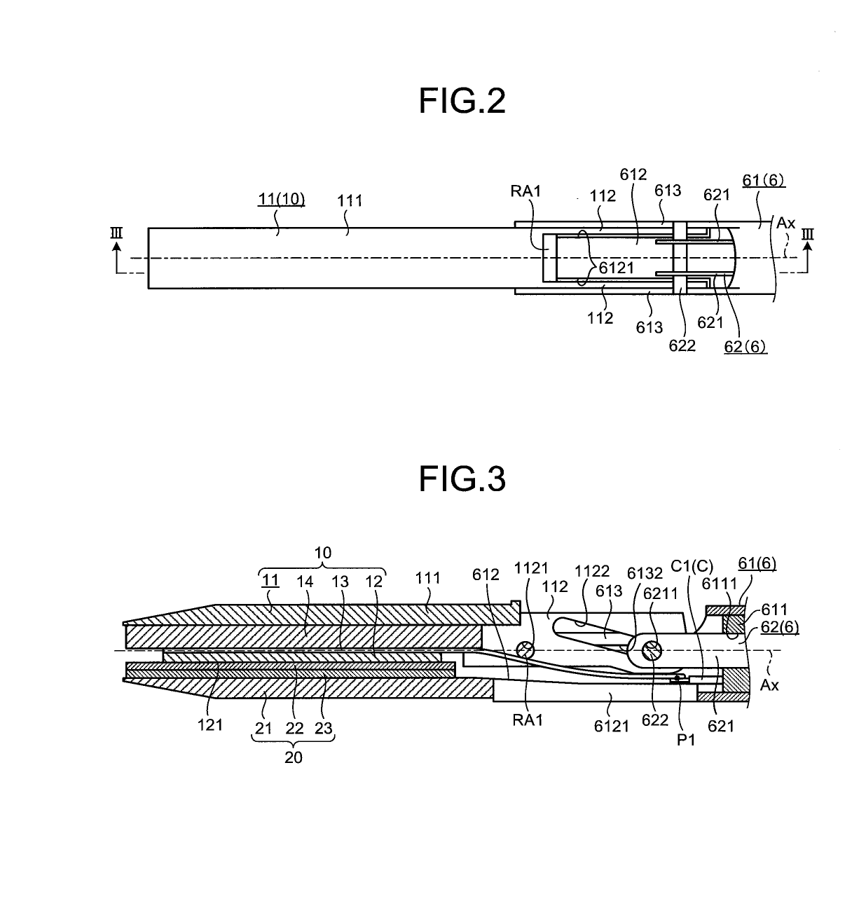

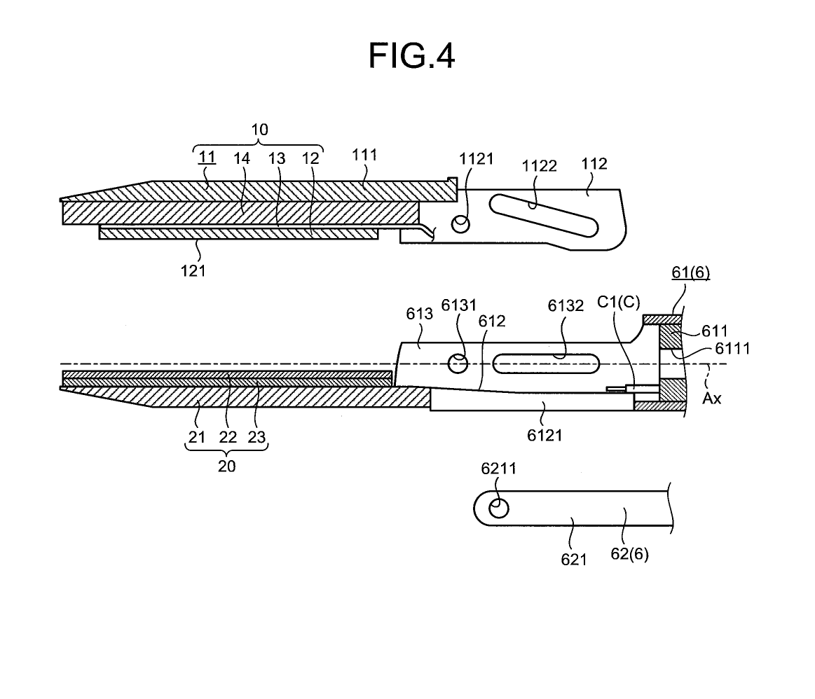

FIGS. 2 to 4 are diagrams illustrating a distal end part of the medical treatment device 2. Specifically, FIG. 2 is a diagram illustrating the distal end part of the medical treatment device 2 from above in FIG. 1. A flexible substrate 13 and a lead wire C1 are not illustrated in FIG. 2. FIG. 3 is a cross-sectional view taken along a line of FIG. 2. The line of FIG. 2 is a line which passes through a gap (a slit 6121) between a lower plate body 621 and a connection portion 112 in FIG. 2. FIG. 4 is an exploded view illustrating a state where a first holding member 10 and a rod 62 are separated from a main shaft body 61 from a state illustrated in FIG. 3. A first rotation axis RA1 and a shaft portion 622 are not illustrated in FIG. 4.

As illustrated in FIGS. 2 to 4, the shaft 6 includes a main shaft body 61, a rod 62, and a cylindrical sheath 63 (FIG. 1) which covers an outer periphery of the main shaft body 61.

The sheath 63 is not illustrated in FIGS. 2 to 4.

The main shaft body 61 has a substantially cylindrical shape, one end thereof is connected to the operation handle 5, and the other end thereof axially supports the first and second holding members 10 and 20 (FIGS. 1 to 4) constituting the grasping portion 7 so that the first and second holding members can be opened and closed.

As illustrated in FIG. 3 or 4, a case 611 is disposed inside the main shaft body 61.

As illustrated in FIG. 3 or 4, the case 611 has a cylindrical shape with a guide hole 6111 extending along the center axis Ax of the main shaft body 61.

Further, although not illustrated in the drawings, the case 611 is provided with a wiring hole which extends along the center axis Ax of the main shaft body 61 in addition to the guide hole 6111. Then, an electric cable C (FIG. 1) which is connected to the control device 3 passes through the operation handle 5 and the wiring hole of the case 611 to be disposed inside the main shaft body 61 from one end side of the main shaft body 61 toward the other end side thereof. A part of the lead wire C1 constituting the electric cable C is illustrated in FIGS. 3 and 4.

Further, as illustrated in FIGS. 2 to 4, the other end of the main shaft body 61 is provided with a base portion 612 and a pair of axial supporters 613.

The base portion 612 has a substantially elongated flat plate shape. Then, the base portion 612 is integrally formed with the lower side of the other end of the main shaft body 61 in FIG. 3 or 4 while plate surfaces are directed upward and downward in FIG. 3 or 4 and the longitudinal direction follows the center axis Ax.

As illustrated in FIG. 2, the base portion 612 has a pair of slits 6121 penetrating the base portion 612 and extending along the center axis Ax, at both sides of the base portion 612 in the width direction.

The pair of axial supporters 613 has a substantially elongated flat plate shape each other. Then, the pair of axial supporters 613 is integrally formed with an upper surface (in FIG. 3 or 4, an upper surface) of the base portion 612 to face each other while the pair of slits 6121 is interposed therebetween and the longitudinal direction follows the center axis Ax.

The pair of axial supporters 613 has the same shape. For this reason, hereinafter, a shape of the upper axial supporter 613 in FIG. 2 will be described.

As illustrated in FIG. 4, the axial supporter 613 has a first bearing hole 6131 penetrating the axial supporter 613, at a location closer to a distal end side (on the left side in FIG. 4) than a center position in the longitudinal direction of the axial supporter 613.

The first bearing hole 6131 is a hole into which the first rotation axis RA1 (FIG. 3) is inserted.

As illustrated in FIG. 4, the axial supporter 613 has a first track hole 6132 penetrating the axial supporter 613 and extending along the center axis Ax, at a location closer to a proximal end side (on the right side in FIG. 4) than the first bearing hole 6131.

The rod 62 is disposed inside the case 611 and moves forward and backward along the center axis Ax in response to the operation of the operation knob 51 from the operator. That is, the rod 62 constitutes a part of an opening and closing mechanism for opening and closing the first and second holding members 10 and 20. As illustrated in FIGS. 2 to 4, the rod 62 includes the pair of plate bodies 621 and the shaft portion 622 (FIGS. 2 and 3).

The pair of plate bodies 621 has an elongated flat plate shape and is disposed inside the case 611 to face each other (to face each other in the vertical direction in FIG. 2). Each of the pair of plate bodies 621 has an insertion hole 6211 (FIGS. 3 and 4) which penetrates each plate body 621 and through which the shaft portion 622 is inserted, at a distal end side of each of the pair of plate bodies 621 (on the left end side in FIGS. 2 to 4).

The shaft portion 622 has a columnar shape and is inserted into each insertion hole 6211 of each of the pair of plate bodies 621. Then, both ends of the shaft portion 622 protrude outward from the pair of plate bodies 621 in a state where the shaft portion 622 is inserted into each insertion hole 6211 as illustrated in FIG. 2. Further, both ends of the, shaft portion 622 protruding outward from the pair of plate bodies 621 are inserted into the first track holes 6132 of the pair of axial supporters 613 and a second track hole 1122 (FIGS. 3 and 4) of a first jaw 11 constituting the first holding member 10.

Configuration of Grasping Portion

The grasping portion 7 is a portion which grasps a body tissue and performs a treatment (for example, a blood vessel sealing or disserting) on the body tissue. As illustrated in FIG. 3 or 4, the grasping portion 7 includes the first holding member 10 and the second holding member 20.

The first holding member 10 is disposed with the second holding member 20 in FIG. 3 or 4. As illustrated in FIGS. 2 to 4, the first holding member 10 includes the first jaw 11, a heat transfer plate 12 (FIGS. 3 and 4), the flexible substrate 13 (FIGS. 3 and 4), and a first fixed plate 14 (FIGS. 3 and 4).

The first jaw 11 is a portion which is axially supported by the pair of axial supporters 613 through the first rotation axis RA1 to be rotatable. As illustrated in FIGS. 2 to 4, the first jaw 11 includes a main jaw 111 and the pair of connection portions 112.

As illustrated in FIG. 2, the main jaw 111 has an elongated flat plate shape in which a width dimension (a length dimension in the width direction) is slightly smaller than a separated dimension of the pair of axial supporters 613.

The pair of connection portions 112 is a portion which connects the first jaw 11 to the main shaft body 61. Each of the pair of connection portions 112 has a substantially elongated flat plate shape. Then, the pair of connection portions 112 is integrally formed with one end side (in FIG. 3 or 4, a right end side) of the main jaw 111 to face each other and to be orthogonal to the main jaw 111 so that the longitudinal direction follows the longitudinal direction of the main jaw 111. As illustrated in FIG. 2, a separated dimension of the pair of connection portions 112 is set to be substantially the same as a width dimension of the main jaw 111. Further, each of the pair of connection portions 112 is formed so that a thickness dimension is slightly smaller than a width dimension of the slit 6121.

The pair of connection portions 112 has the same shape. For this reason, hereinafter, a shape of the upper connection portion 112 in FIG. 2 will be described.

As illustrated in FIG. 3 or 4, the connection portion 112 has a second bearing hole 1121 penetrating the connection portion 112, at a location closer to a proximal end side (on the left side in FIG. 3 or 4) than a center position in the longitudinal direction of the connection portion 112.

The second bearing hole 1121 is a hole into which the first rotation axis RA1 (FIGS. 2 and 3) is inserted. That is, when the pair of connection portions 112 is positioned between the pair of axial supporters 613 and the first rotation axis RA1 is inserted into each of the first bearing holes 6131 and each of the second bearing holes 1121, the first jaw 11 is axially supported by the main shaft body 61 (the pair of axial supporters 613) to be rotatable about the first rotation axis RA1.

As illustrated in FIG. 3 or 4, the connection portion 112 has the second track hole 1122 penetrating the connection portion 112 and extending in a direction intersecting with the center axis Ax, at a location closer to a distal end side (on the right side in FIG. 4) than the second bearing hole 1121.

Specifically, the second track hole 1122 has a shape which is inclined upward in FIG. 3 or 4 as it goes toward the second bearing hole 1121. In a state illustrated in FIG. 3 (a state where the first and second holding members 10 and 20 are closed), a right end of the second track hole 1122 in FIG. 3 is set so that a height position (a height position from the base portion 612) is the same as that of a right end of a first track hole 6112 in FIG. 3. That is, in a state illustrated in FIG. 3, the second track hole 1122 is set so that a height position gradually increases with respect to the first track hole 6132 as it goes toward the second bearing hole 1121. Then, the end of the shaft portion 622 is inserted into the second track hole 1122.

The heat transfer plate 12 is formed by, for example, a thin copper plate and is attached to the first jaw 11 such that a treatment surface 121 (FIGS. 3 and 4) as one plate surface faces the second holding member 20 when the first jaw 11 is connected to the main shaft body 61. Then, in a state where a body tissue is grasped by the first and second holding members 10 and 20, the heat transfer plate 12 brings the treatment surface 121 into contact with the body tissue and transfers heat from the flexible substrate 13 to the body tissue (so that energy (heat energy) is applied to the body tissue). That is, the heat transfer plate 12 serves as an energy application portion according to the invention.

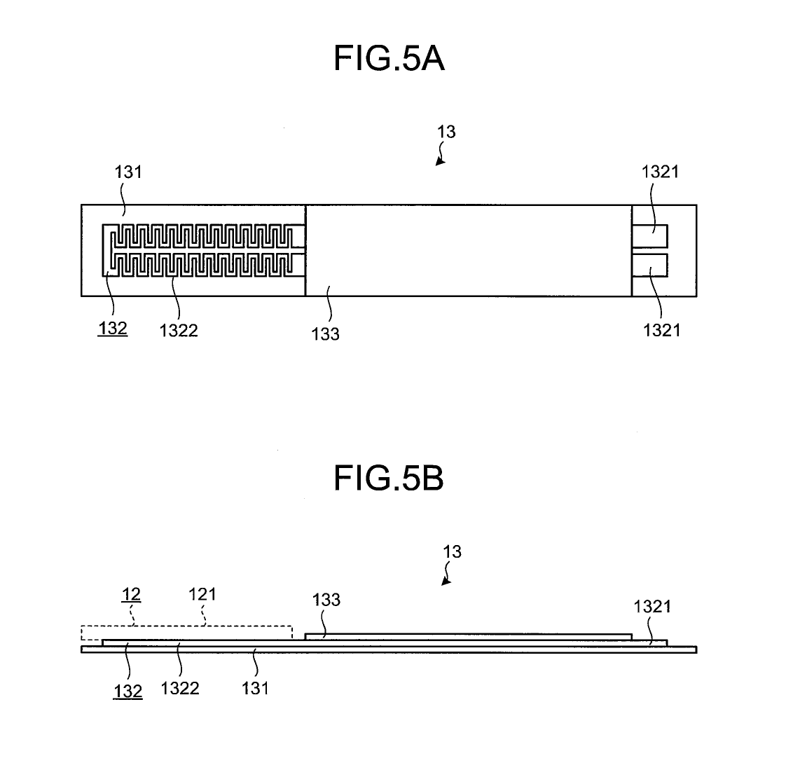

FIGS. 5A and 5B are diagrams illustrating the flexible substrate 13. Specifically, FIG. 5A is a top view illustrating the flexible substrate 13 illustrated in FIG. 3 or 4 when viewed from below in FIG. 3 or 4. FIG. 5B is a side view of the flexible substrate 13.

The flexible substrate 13 serves as a sheet heater which generates heat from a part thereof and heats the heat transfer plate 12 by the heat. As illustrated in FIG. 5A or 5B, the flexible substrate 13 includes a substrate 131, a heating pattern 132, and an insulation sheet 133.

The substrate 131 is an elongated sheet which is formed of an insulation material such as polyimide. A width dimension of the substrate 131 (in FIG. 5A, a length dimension in the vertical direction) is set to be slightly smaller than the separated dimension of the pair of connection portions 112. Then, a part of the flexible substrate 13 is disposed between the pair of connection portions 112 while being attached to the first jaw 11.

The heating pattern 132 is obtained by processing a metal film formed on one surface of the substrate 131 by adhering or depositing and is used to heat the heat transfer plate 12. As illustrated in FIG. 5A or 5B, the heating pattern 132 includes a pair of lead connection portions 1321 and an electric resistance pattern 1322.

Here, a material of the heating pattern 132 is stainless steel or platinum.

The pair of lead connection portions 1321 extends from one end side (in FIG. 5A or 5B, a right end side) of the substrate 131 toward the other end side (in FIG. 5A or 5B, a left end side) and is provided to face each other. Then, two lead wires C1 constituting the electric cable C are bonded (connected) to the pair of lead connection portions 1321 (see FIG. 3 (in FIG. 3, only one lead wire C1 is illustrated).

One end of the electric resistance pattern 1322 is connected to one lead connection portion 1321, a U-shape is formed from the one end to follow an outer edge shape of the substrate 131, and the other end thereof is connected to the other lead connection portion 1321. Then, the electric resistance pattern 1322 generates heat when a voltage is applied (supplied) to the pair of lead connection portions 1321 through two lead wires C1 under the control of the control device 3.

Then, as illustrated in FIG. 5A, the heat transfer plate 12 is attached to a portion (a heating portion) provided with the electric resistance pattern 1322 in the flexible substrate 13. Although not illustrated in detail in drawings, an adhesive sheet which causes the heat transfer plate 12 and the flexible substrate 13 to adhere to each other is interposed between the heat transfer plate 12 and the flexible substrate 13. The adhesive sheet is a sheet having high thermal conductivity, high-temperature resistance, and adhesiveness and is formed by mixing ceramic having high thermal conductivity such as alumina or aluminum nitride with, for example, epoxy resin.

Similar to the substrate 131, the insulation sheet 133 is an elongated sheet which is formed of an insulation material such as polyimide. Then, as illustrated in FIG. 5A or 5B, the insulation sheet 133 is attached to cover the pair of lead connection portions 1321 except for a part of the pair of lead connection portions 1321 (in FIG. 5A or 5B, a right end).

The first fixed plate 14 is a member that fixes a unit, fixing the heat transfer plate 12 to the flexible substrate 13, to one plate surface (in FIG. 3 or 4, a lower plate surface) of the main jaw 111.

As illustrated in FIG. 3 or 4, the second holding member 20 includes a second jaw 21, a grasping plate 22, and a second fixed plate 23.

The second jaw 21 has the substantially same shape as that of the main jaw 111. Then, in the first embodiment, as illustrated in FIG. 3 or 4, the second jaw 21 is integrally formed with a distal end part (in FIG. 3 or 4, a left end) of the base portion 612 while plate surfaces are directed upward and downward in FIG. 3 or 4 and the longitudinal direction follows the center axis Ax of the main shaft body 61.

The grasping plate 22 is a member that is formed by, for example, a thin copper plate and grasps a body tissue between the grasping plate and the heat transfer plate 12.

The second fixed plate 23 is a member that fixes the grasping plate 22 to one plate surface (in FIG. 3 or 4, an upper plate surface) of the second jaw 21.

Operation of Opening and Closing First and Second Holding Members

Next, an operation of opening and closing the first and second holding members 10 and 20 will be described.

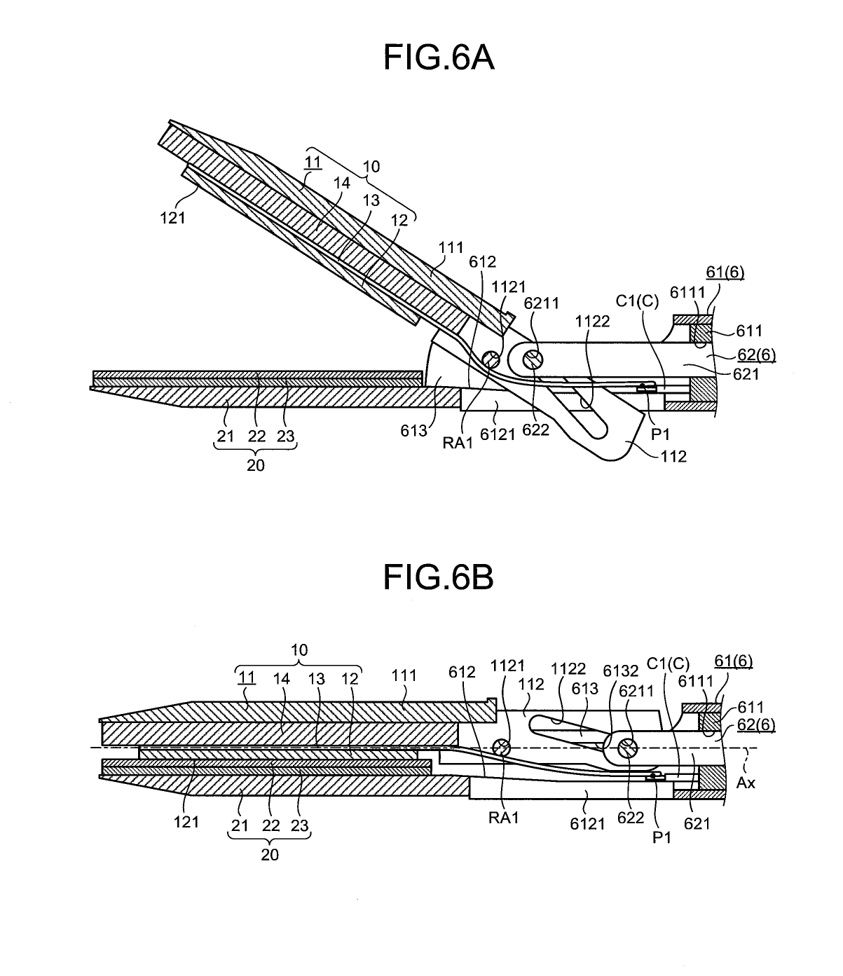

FIGS. 6A and 6B are diagrams illustrating an operation of opening and closing the first and second holding members 10 and 20. Specifically, FIG. 6A is a cross-sectional view corresponding to FIG. 3 and is a diagram illustrating a state where the first and second holding members 10 and 20 are opened. FIG. 6B is a cross-sectional view corresponding to FIG. 3 and is a diagram illustrating a state where the first and second holding members 10 and 20 are closed.

FIG. 6A illustrates a state where the operation knob 51 is not operated by the operator. In this state, as illustrated in FIG. 6A, the first and second holding members 10 and 20 are opened.

When the operation knob 51 is operated by the operator from a state illustrated in FIG. 6A, the rod 62 moves toward the operating unit 5 (in FIG. 6A or 6B, a right side). By the movement of the rod 62, the shaft portion 622 moves inside each of the first track holes 6132 and each of the second track holes 1122 from a left side toward a right side in FIG. 6A or 6B.

Here, each of the first track holes 6132 provided in the main shaft body 61 extends along the center axis Ax as described above. Meanwhile, as described above, each of the second track holes 1122 provided in the second jaw 21 is formed so that a height position gradually increases with respect to each of the first track holes 6132 as it goes to a left side in FIG. 6A or 6B.

For this reason, in a case where the shaft portion 622 moves inside each of the first track holes 6132 and each of the second track holes 1122 from a left side toward a right side in FIG. 6A or 6B, the shaft portion moves while pressing edges of the second track holes 1122. Then, the first holding member 10 rotates about the first rotation axis RA1 in a direction moving close to the second holding member 20 and finally becomes a state illustrated in FIG. 6B. At this time, the pair of connection portions 112 is inserted into the pair of slits 6121.

When the operation of the operation knob 51 from the operator is canceled from a state illustrated in FIG. 6B, the rod 62 moves from a right side toward a left side in FIG. 6A or 6B in a manner opposite to the above-described order. Then, in accordance with the movement of the rod 62, the first holding member 10 rotates about the first rotation axis RA1 in a direction moving away from the second holding member 20 in a manner opposite to the above-described order and finally becomes a state illustrated in FIG. 6A.

Connection Position between Flexible Substrate and Lead Wire

Next, a connection position P1 between the flexible substrate 13 and the lead wire Cl will be described with reference to FIGS. 6A and 6B.

As illustrated in FIG. 6A or 6B, the flexible substrate 13 is connected to the lead wire C1 at a location closer to one end of the main shaft body 61 (on the right side in FIG. 6A or 6B) than the first rotation axis RA1.

More specifically, as illustrated in FIG. 6A, the connection position P1 between the flexible substrate 13 and the lead wire C1 is closer to one end of the main shaft body 61 than a position of the shaft portion 622 when the first and second holding members 10 and 20 are closed.

Configuration of Control Device and Foot Switch

The foot switch 4 is operated by a foot from the operator. Then, the medical treatment device 2 (the electric resistance pattern 1322) is turned on and off by the control device 3 in response to the operation of the foot switch 4.

Means for switching an on/off state is not limited to the foot switch 4 and may be a switch which is operated by a hand.

The control device 3 includes a CPU (Central Processing Unit) and the like to control an operation of the medical treatment device 2 in accordance with a predetermined control program. More specifically, the control device 3 applies a voltage to the electric resistance pattern 1322 through the electric cable C in response to an operation (a power on/off operation) of the foot switch 4 from the operator, thereby to heat the heat transfer plate 12.

Operation of Medical Treatment System

Next, an operation (an operation method) of the medical treatment system 1 will be described.

The operator grips the medical treatment device 2 and inserts a distal end part (a part of the grasping portion 7 and the shaft 6) of the medical treatment device 2 into, for example, an abdominal cavity through an abdominal wall. Then, the operator operates the operation knob 51 to rotate the first holding member 10 such that a body tissue of a treatment object is pinched by the first and second holding members 10 and 20.

Next, the operator operates the foot switch 4 to select a power on state in which power is supplied from the control device 3 to the medical treatment device 2. When the power is on, the control device 3 applies a voltage to the electric resistance pattern 1322 through the electric cable C to heat the heat transfer plate 12. Then, a body tissue which contacts the heat transfer plate 12 is treated by the heat of the heat transfer plate 12 (for example, a blood vessel is sealed or disserting of a blood vessel is performed).

In the medical treatment device 2 according to the first embodiment described above, wiring inside the medical treatment device 2 is formed by a combination of the flexible substrate 13 and the lead wire C1. For this reason, a manufacturing cost of the medical treatment device 2 can be decreased compared to a configuration in which all the wirings inside the device 2 are formed by the flexible substrate.

In the medical treatment device 2 according to the first embodiment, the connection position P1 between the flexible substrate 13 and the lead wire C1 is closer to one end of the main shaft body 61 than a position of the shaft portion 622 (closer to one end of the main shaft body 61 than the first rotation axis RA1) when the first and second holding members 10 and 20 are closed. In other words, the connection position P1 is closer to one end of the main shaft body 61 than the first rotation axis RA1 so as to avoid an area which may contact the opening and closing mechanism (the shaft portion 622) when the first and second holding members 10 and 20 are opened and closed (the area between the position of the shaft portion 622 when the first and second holding members 10 and 20 are opened and the position of the shaft portion 622 when the first and second holding members 10 and 20 are closed). For this reason, it is possible to prevent interference between the connection position P1 and the first rotation axis RA1 or the shaft portion 622 when the first and second holding members 10 and 20 are opened and closed. Thus, since a load applied to the connection position 21 is reduced, disconnection can be inhibited.

From the description above, according to the medical treatment device 2 of the first embodiment, disconnection can be inhibited while an increase in manufacturing cost is prevented.

### MODIFIED EXAMPLE 1-1 OF FIRST EMBODIMENT

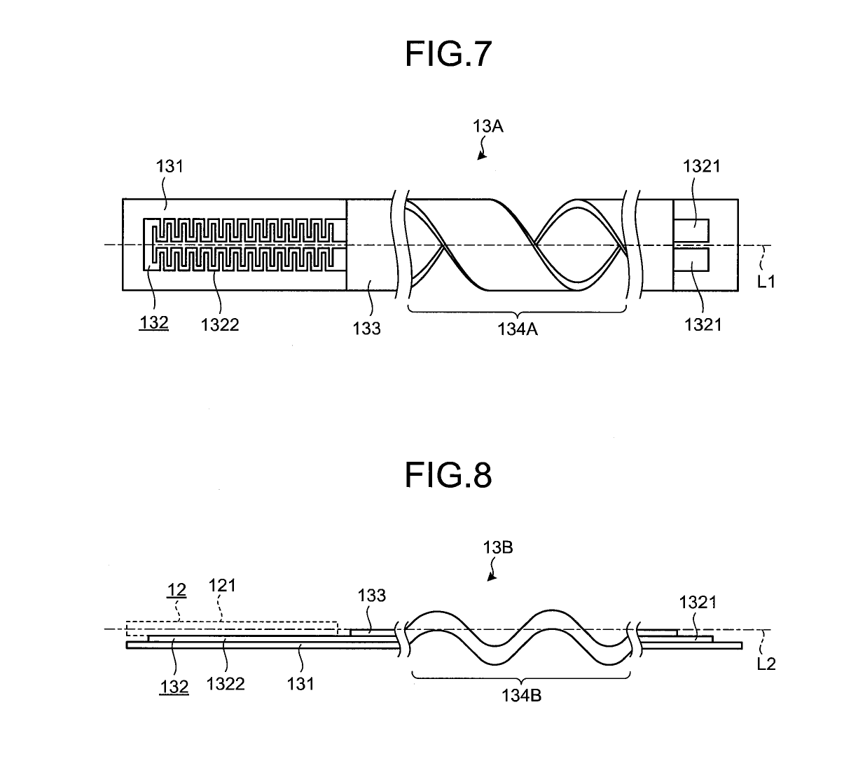

FIG. 7 is a diagram illustrating Modified Example 1-1 of the first embodiment of the invention. Specifically, FIG. 7 corresponds to FIG. 5A and is a diagram illustrating a flexible substrate 13A according to Modified Example 1-1.

The flexible substrate 13 according to the first embodiment has a substantially flat plate shape, but the invention is not limited thereto. The flexible substrate 13A including an elastic portion 134A according to Modified Example 1-1 may be employed.

The flexible substrate 13A has the same configuration as that of the flexible substrate 13 described in the first embodiment except for the elastic portion 134A.

The elastic portion 134A is a part of the flexible substrate 13A (the part where the insulation sheet 133 is provided) and has a flexure shape. The flexible substrate 13A is extensible by the shape.

Specifically, as illustrated in FIG. 7, the elastic portion 134A is formed in a spiral (a flexure shape) about the longitudinal direction of the flexible substrate 13A (a virtual line L1 directed from the other end side toward one end side of the main shaft body 61 (a line which is parallel to the center axis Ax)).

Although not illustrated in detail in the drawings, the elastic portion 134A is disposed between the first rotation axis RA1 and the connection position (for example, the connection position P1 illustrated in FIGS. 6A and 6B) between the flexible substrate 13A and the lead wire C1. For example, the elastic portion 134A is wound at a location closer to a proximal end side (on the right side in FIG. 6A or 6B) than the shaft portion 622 of the rod 62 (the pair of plate bodies 621).

When the elastic portion 134A is provided in the flexible substrate 13A as in Modified Example 1-1, the elastic portion 134A can absorb a tension applied to the flexible substrate 13A when the first and second holding members 10 and 20 are opened and closed. Thus, it is possible to further reduce a load applied to the connection position between the flexible substrate 13A and the lead wire C1.

### MODIFIED EXAMPLE 1-2 OF FIRST EMBODIMENT

FIG. 8 is a diagram illustrating Modified Example 1-2 of the first embodiment of the invention. Specifically, FIG. 8 corresponds to FIG. 5B and is a diagram illustrating a flexible substrate 13B according to Modified Example 1-2.

The flexible substrate 13 according to the first embodiment has a substantially flat plate shape, but the invention is not limited thereto. The flexible substrate 13B including an elastic portion 134B according to Modified Example 1-2 may be employed.

The flexible substrate 13A has the same configuration as that of the flexible substrate 13 described in the first embodiment except for the elastic portion 134B.

The elastic portion 134B is a part of the flexible substrate 13B (the part where the insulation sheet 133 is provided) and has a flexure shape. The flexible substrate 13B is extensible by the shape.

Specifically, as illustrated in FIG. 8, the elastic portion 134B has a wave shape (a flexure shape) to intersect with the longitudinal direction of the flexible substrate 13B (a virtual line L2 directed from the other end toward one end of the main shaft body 61 (a line which is parallel to the center axis Ax)).

Then, although not illustrated in detail in drawings, the elastic portion 134B is disposed between the first rotation axis RA1 and the connection position (for example, the connection position 21 illustrated in FIGS. 6A and 6B) between the flexible substrate 13B and the lead wire C1.

When the elastic portion 134B is provided in the flexible substrate 13B as in Modified Example 1-2, the same effect as that of Modified Example 1-1 can be obtained.

### MODIFIED EXAMPLE 1-3 OF FIRST EMBODIMENT

In the first embodiment, a portion of the flexible substrate 13 which is closer to one end of the main shaft body 61 than the first rotation axis RA1 (for example, a portion of the connection position P1 between the flexible substrate 13 and the lead wire C1) may be fixed to the shaft 6. The same also applies to Modified Examples 1-1 and 1-2.

As in Modified Example 1-3, if the portion of the flexible substrate 13 which is closer to the one end of the main shaft body 61 than the first rotation axis RA1 is fixed to the shaft 6, it is possible to further reduce the load applied to the connection position P1.

### Second Embodiment

Next, a second embodiment of the invention will be described.

In the description below, the same reference signs will be used to designate the same elements as those of the first embodiment, and a detailed explanation thereof will be omitted or simplified.

A medical treatment device according to the second embodiment is different from the medical treatment device 2 described in the first embodiment in that the configuration of the main shaft body 61 is different. For this reason, hereinafter, a configuration of the shaft body according to the second embodiment will be described.

Configuration of Shaft Body

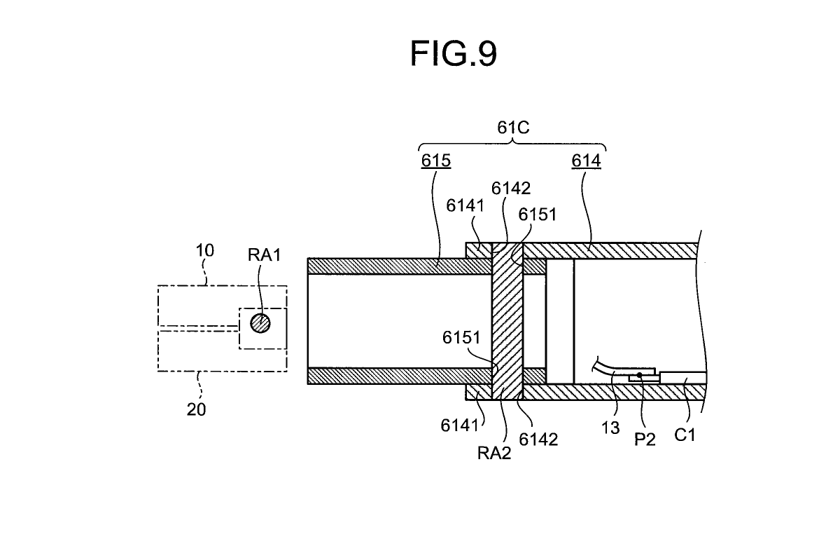

FIG. 9 is a cross-sectional view illustrating a configuration of a main shaft body 61C according to the second embodiment of the invention.

For the purpose of illustration, a configuration of the other end of the main shaft body 61C (including the first and second holding members 10 and 20) is simply illustrated in FIG. 9. Further, a sheath covering the outer periphery of the main shaft body 61C is also not illustrated in the drawings.

Similar to the first embodiment, one end (in FIG. 9, a right end) of the main shaft body 61C is connected to the operation handle 5 and the other end (in FIG. 9, a left end) thereof axially supports the first and second holding members 10 and 20 so that the first and second holding members can be opened and closed about the first rotation axis RA1.

As illustrated in FIG. 9, the main shaft body 61C includes first and second shafts 614 and 615 each having a substantially cylindrical shape.

The first shaft 614 is a portion which has the above-described end and is connected to the operation handle 5.

The first shaft 614 has a pair of rotation receiving portions 6141 connected to the second shaft 615, at a distal end (at a left end in FIG. 9) of the first shaft 614.

Each of the pair of rotation receiving portions 6141 has a substantially flat plate shape. The pair of rotation receiving portions 6141 is integrally formed with a distal end of the first shaft 614 to face each other while plate faces are directed upward and downward in FIG. 9.

Each of the pair of rotation receiving portions 6141 has a third bearing hole 6142 which penetrates each rotation receiving portion and into which the second rotation axis RA2 is inserted.

The second shaft 615 is a portion which includes the above-described other end and axially supports the first and second holding members by the other end so that the first and second holding members can be opened and closed about the first rotation axis RA1. Then, the second shaft 615 is formed in a substantially cylindrical shape having an outer diameter dimension which is slightly smaller than a separated dimension of the pair of rotation receiving portions 6141.

The second shaft 615 has, at a proximal end thereof (on the right end side in FIG. 9), a pair of fourth bearing holes 6151 into which the second rotation axis RA2 is inserted. That is, the proximal end of the second shaft 615 is located between the pair of rotation receiving portions 6141, and the second rotation axis RA2 is inserted into each of the third bearing holes 6142 and each of the fourth bearing holes 6151. With this structure, the second shaft 615 is axially supported by the first shaft 614 (the pair of rotation receiving portions 6141) so as to be rotatable about the second rotation axis RA2.

Here, as illustrated in FIG. 9, the second rotation axis RA2 is an axis which is orthogonal to the first rotation axis RA1.

Although not illustrated in detail in drawings, the inside of the first shaft 614 is provided with an opening and closing mechanism which opens and closes the first and second holding members 10 and 20 or a rotating mechanism which rotates the second shaft 615 about the second rotation axis RA2 in response to the operation of the operation handle 5 from the operator.

Connection Position between Flexible Substrate and Lead Wire

Next, a connection position P2 between the flexible substrate 13 and the lead wire Cl of the second embodiment will be described with reference to FIG. 9.

In the second embodiment, as illustrated in FIG. 9, the connection position P2 is located closer to one end of the main shaft body 61C (on the right side in FIG. 9) than the second rotation axis RA2.

According to the above-described embodiments, the following effects can be obtained in addition to the same effect as that of the first embodiment.

In the second embodiment, the connection position P2 between the flexible substrate 13 and the lead wire C1 is closer to the one end of the main shaft body 61C than the second rotation axis RA2. For this reason, even when the main shaft body 61C has a structure in which the second shaft 615 is rotatable with respect to the first shaft 614, it is possible to prevent an interference between the connection position P2 and the first rotation axis RA1 or the second rotation axis RA2 when the first and second holding members 10 and 20 are opened and closed or the second shaft 615 rotates with respect to the first shaft 614. Thus, since a load applied to the connection position P2 is reduced, disconnection can be inhibited.

### MODIFIED EXAMPLE 2-1 OF SECOND EMBODIMENT

In the second embodiment, the flexible substrate 13A (FIG. 7) described in Modified Example 1-1 may be employed instead of the flexible substrate 13. In this way, when the flexible substrate 13A is employed, the elastic portion 134A is disposed between the first and second rotation axes RA1 and RA2.

When the elastic portion 134A is provided in the flexible substrate 13A as in Modified Example 2-1, it is possible to absorb a tension applied to the flexible substrate 13A by the elastic portion 134A when the first and second holding members 10 and 20 are opened and closed or the second shaft 615 rotates with respect to the first shaft 614. Thus, it is possible to further reduce a load applied to the connection position P2 between the flexible substrate 13A and the lead wire C1.

### MODIFIED EXAMPLE 2-2 OF SECOND EMBODIMENT

In the second embodiment, the flexible substrate 13B (FIG. 8) described in Modified Example 1-2 may be employed instead of the flexible substrate 13. In this way, even when the flexible substrate 13B is employed, the elastic portion 134B is disposed between the first and second rotation axes RA1 and RA2.

When the elastic portion 134B is provided in the flexible substrate 13B as in Modified Example 2-2, the same effect as that of Modified Example 2-1 can be obtained.

### MODIFIED EXAMPLE 2-3 OF SECOND EMBODIMENT

In the second embodiment, a portion of the flexible substrate 13 which is closer to one end of the main shaft body 61C (for example, a portion of the connection position P2) than the second rotation axis RA2 may be fixed to the main shaft body 61C (the first shaft 614). The same also applies to Modified Examples 2-1 and 2-2.

As in Modified Example 2-3, if the portion of the flexible substrate 13 which is closer to the one end of the main shaft body 61C than the second rotation axis RA2 is fixed to the main shaft body 61C, it is possible to further reduce a load applied to the connection position P2.

### Other Embodiments

Although the embodiments of the invention have been described so far, the invention is not limited to the first embodiment and the second embodiment or Modified Examples 1-1 to 1-3 and 2-1 to 2-3.

In the first embodiment and the second embodiment or Modified Examples 1-1 to 1-3 and 2-1 to 2-3, a configuration (the heat transfer plate 12 and the flexible substrate 13, 13A, 13B) of applying energy (heat energy) to a body tissue only from the first holding member 10 has been employed, but the invention is not limited thereto. For example, energy may be applied to a body tissue only from the second holding member 20 or energy may be applied to a body tissue from both the first and second holding members 10 and 20.

In the first embodiment and the second embodiment or Modified Examples 1-1 to 1-3 and 2-1 to 2-3, the first and second holding members 10 and 20 are opened and closed in such a manner that the second holding member 20 is fixed and the first holding member 10 is rotated, but the invention is not limited thereto. For example, the first and second holding members 10 and 20 may be opened and closed in such a manner that the first holding member 10 is fixed and the second holding member 20 is rotated or the first and second holding members 10 and 20 may be opened and closed in such a manner that both the first and second holding members 10 and 20 are rotated.

Further, an opening and closing mechanism of opening and closing the first and second holding members 10 and 20 or a rotating mechanism of rotating the second shaft 615 with respect to the first shaft 614 is not limited to the opening and closing mechanism or the rotating mechanism described in the first embodiment and the second embodiment or Modified Examples 1-1 to 1-3 and 2-1 to 2-3 and the other mechanisms may be employed.

In the first embodiment and the second embodiment or Modified Examples 1-1 to 1-3 and 2-1 to 2-3, heat energy is employed as energy applied to a body tissue, but high-frequency energy or ultrasonic energy may be employed. Further, a combination of two or more different energies among the heat energy, the high-frequency energy, and the ultrasonic energy may be sequentially applied to a body tissue.

According to the medical treatment device of some embodiments, it is possible to prevent an increase in manufacturing cost as well as to suppress disconnection.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical treatment device comprising:
a pair of holding members configured to open and close about a first rotation axis in accordance with an operation of an operating unit; and
a shaft having one end connected to the operating unit and having the other end to axially support the pair of holding members through the first rotation axis so as to allow the pair of holding members to open and close, wherein
at least one of the pair of holding members comprises an energy application portion configured to contact a body tissue to apply energy to the body tissue with the body tissue grasped by the pair of holding members, wherein
the energy application portion is connected to a flexible substrate configured to be electrically connected to a lead wire provided inside the shaft extending from the other end of the shaft toward the one end of the shaft to supply energy to the energy application portion, wherein
the flexible substrate is connected to the lead wire at a location closer to the one end of the shaft than the first rotation axis.

2. The medical treatment device according to claim 1, wherein
a part of the flexible substrate comprises an elastic portion having a flexure shape to allow the elastic portion to be extensible, and
the elastic portion is disposed between the first rotation axis and a connection position, and between the flexible substrate and the lead wire.

3. The medical treatment device according to claim 2, wherein
the elastic portion is formed in a spiral shape about a virtual line directed from the other end toward the one end of the shaft.

4. The medical treatment device according to claim 2, wherein
the elastic portion is formed in a wave shape to intersect with a virtual line directed from the other end toward the one end of the shaft.

5. The medical treatment device according to claim 1, wherein
a part of the flexible substrate is fixed to the shaft at a location closer to the one end of the shaft than the first rotation axis.

6. The medical treatment device according to claim 1, wherein
the shaft comprises:
a first shaft having the one end; and
a second shaft having the other end and connected to the first shaft so as to be rotatable about a second rotation axis, wherein
the flexible substrate is connected to the lead wire at a location closer to the one end of the first shaft than the second rotation axis.

7. The medical treatment device according to claim 6, wherein
a part of the flexible substrate comprises an elastic portion having a flexure shape to allow the elastic portion to be extensible, and
the elastic portion is disposed between the first rotation axis and the second rotation axis.
